(19) United States
(12) Patent Application Publication
(10) Pub. No.: US 2026/0215719 A1
(43) Pub. Date: Jul. 30, 2026

(54) ONE MEANS OF PATIENT PROTECTION ("MOPP") STRAIN RELIEF

(71) Applicant: Draeger Medical Systems, Inc., Andover, MA (US)

(72) Inventor: Nicholas Comei, Bradford, MA (US)

(21) Appl. No.: 19/456,878

(22) Filed: Jan. 22, 2026

Related U.S. Application Data
(60) Provisional application No. 63/750,162, filed on Jan. 27, 2025.

(51) Int. Cl. A61B 5/301; H01R 13/58; H01R 13/66

(52) U.S. Cl. CPC A61B 5/301; H01R 13/5845; H01R 13/665; A61B 2562/222; H01R 2201/12

(57) ABSTRACT

The present disclosure pertains to providing electrical isolation to a medical patient during medical care that is delivered, at least in part, by, with, or through medical devices. The disclosed technique provides a means of patient protection (“MOPP”) by disposing an electrical isolator in the strain relief of a cable providing electrical connectivity between a host medical device and one or more medical sensors. The strain relief and electrical isolator are disposed at an end of the cable that is most proximate to the host medical device. Electrical connectors are disposed on each end of the cable to provide connection with the host medical device and the one or more sensors.

### CROSS REFERENCE TO RELATED APPLICATION

Not applicable.

STATEMENT REGARDING FEDERALLY SPONSORED RESEARCH OR DEVELOPMENT

Not applicable.

### TECHNICAL FIELD

The present disclosure pertains to providing electrical isolation to a medical patient during medical care that is delivered, at least in part, by, with, or through medical devices.

### DESCRIPTION OF THE RELATED ART

This section of this document introduces information about and/or from the art that may provide context for or be related to the subject matter described herein and/or claimed below. It provides background information to facilitate a better understanding of the various aspects of the present disclosure. This is a discussion of “related” art. That such art is related in no way implies that it is also “prior” art. The related art may or may not be prior art. The discussion in this section of this document is to be read in this light, and not as admissions of prior art.

Modern medical care employs many types of medical devices to many different ends. A number of design standards apply to such medical devices for a variety of reasons. Some of these standards apply to what are known in the art as “means of patient protection”, or “MOPP”. Various standards may require one or more MOPPs, which may be referred to as, for example, one MOPP, two MOPPs, etc. Some MOPPs are directed to a form of patient electrical safety, including electrical isolation from medical devices.

### SUMMARY

The presently disclosed technique provides a means of patient protection (“MOPP”) by disposing an electrical isolator in the strain relief of a cable providing electrical connectivity between a host medical device and one or more medical sensors. The strain relief and electrical isolator are disposed at an end of the cable that is most proximate to the host medical device. Electrical connectors are disposed on each end of the cable to provide connection with the host medical device and the one or more sensors.

Accordingly, in a first aspect, a cable comprises a plurality of cabled electrical conductors, a first connector, and a strain relief. The cabled electrical conductors having a first end and a second end. The first electrical connector is disposed at the first end thereof and the strain relief is disposed at the second end. The strain relief includes a second electrical connector and providing electrical isolation to the first connector.

In a second aspect, a patient care system comprises a host medical device, a medical sensor, and a cable. The cable electrically connects the host medical device to the medical sensor. The cable comprises a plurality of cabled electrical conductors, a first connector, and a strain relief. The cabled electrical conductors having a first end and a second end. The first electrical connector is disposed at the first end thereof and the strain relief is disposed at the second end. The strain relief includes a second electrical connector and providing electrical isolation to the first connector.

In a third aspect, a method provides electrical isolation to a patient in a patient care system. The method comprises: connecting a cable at a first end thereof to a medical sensor; connecting the cable to a host medical device at a second end thereof opposite the first end; providing strain relief for the connection to the host medical device at the second end of the cable; and providing electrical isolation for the connection to the host medical device external to the host medical device and at the second end of the cable.

In a fourth aspect, a cable is substantially as shown and described herein.

In a fifth aspect, a patient care system is substantially as shown and described herein.

In a sixth aspect, a method for providing electrical isolation to a patient in a patient care system is substantially as shown and described herein.

The above presents a simplified summary of the presently disclosed technique as claimed below in order to provide a basic understanding of some aspects of the presently disclosed technique. This summary is not an exhaustive overview of the presently disclosed technique. It is not intended to identify key or critical elements of the presently disclosed technique or to delineate the scope of the presently disclosed technique. Its sole purpose is to present some concepts in a simplified form as a prelude to the more detailed description that is discussed later.

### BRIEF DESCRIPTION OF THE DRAWINGS

Illustrative embodiments of the claimed subject matter are described herein making reference to the appended drawings.

FIG. 1 is a conceptual block diagram of a patient care system in accordance with one or more embodiments.

FIG. 2A-FIG. 2B are conceptual block diagrams of the strain relief first shown in FIG. 1 in side and top views, respectively.

FIG. 3 is a sectioned view of the cable first shown in FIG. 1 along line 3-3 in FIG. 1.

FIG. 4 depicts one particular embodiment of a patient care system in accordance with one or more embodiments.

While the disclosed subject matter is susceptible to various modifications and alternative forms, the drawings illustrate specific implementations described in detail by way of example. It should be understood, however, that the description herein of specific examples is not intended to limit that which is claimed to the particular forms disclosed, but on the contrary, the intention is to cover all modifications, equivalents, and alternatives falling within the spirit and scope of the appended claims.

### DETAILED DESCRIPTION

The following detailed description is made with reference to the accompanying drawings and is provided to assist in a comprehensive understanding of various example embodiments of the present disclosure. The following description includes various details to assist in that understanding, but these are to be regarded as mere examples. Accordingly, those of ordinary skill in the art will recognize that various changes and modifications of the examples described herein can be made without departing from the spirit and scope of the present disclosure. In addition, descriptions of well-known functions and constructions may be omitted for clarity and conciseness.

Providing electrical patient safety is not always efficient. For example, MOPP measures directed to electrical patient safety generate heat, sometimes to unsafe temperatures for patients, caregivers, and operators. They also can be expensive and take up space in the various medical devices or at the patient's bedside. The most efficient place to provide patient isolation is to have it included as close to the power load as possible—e.g., in the sensor. However, positioning the electrical isolation closer to the sensor also means that this heat source is closer to the patient. Additionally, many medical device makers provide one MOPP in their products while some applications implicate two MOPP. These applications then result in third parties providing additional MOPP or the medical device makers developing a custom solution.

The presently disclosed technique adds one means of protection by taking advantage of the area used for a strain relief for an electrical cable. The technique provides one MOPP in the cable as further described below. The technique thereby provides additional MOPP over and above that provided by a medical device, helps control costs, redistribures heat/power losses, and helps keep excess heat away from patients.

Illustrative examples of the subject matter claimed below are disclosed. In the interest of clarity, not all features of an actual implementation are described for every example in this specification. It will be appreciated that in the development of any such actual implementation, numerous implementation-specific decisions may be made to achieve the developers'specific goals, such as compliance with system-related and business-related constraints, which will vary from one implementation to another. Moreover, it will be appreciated that such a development effort, even if complex and time-consuming, would be a routine undertaking for those of ordinary skill in the art having the benefit of this disclosure.

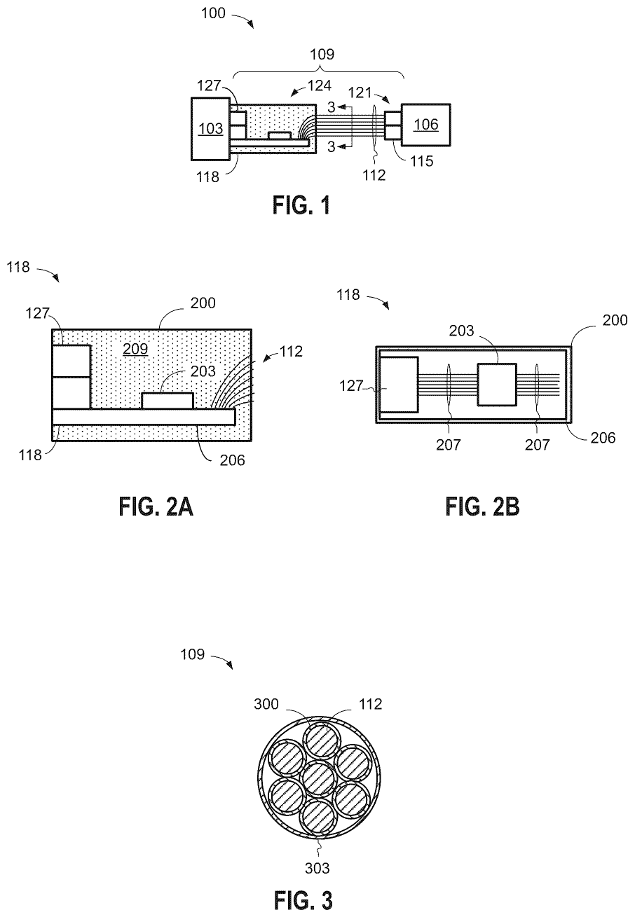

FIG. 1 is a conceptual block diagram of a patient care system 100 in accordance with one or more embodiments. The patient care system 100 includes a host medical device 103, a medical sensor 106, and a cable 109 electrically connecting the host medical device 103 to the medical sensor 106. The cable 109 comprises a plurality of cabled electrical conductors 112, a first electrical connector 115, and a strain relief 118.

The cabled electrical conductors 112, and thus the cable 109 as a whole, each have a distal end 121 and a proximal end 124 relative to the host medical device 102. The first electrical connector 115 is disposed at the distal end 121 and the strain relief 118 is disposed at the proximal end 124. The strain relief 118 includes a second electrical connector 127 and provides electrical isolation to the first connector 115.

As first shown in FIG. 1 but best shown in FIGS. 2A-2B, the strain relief 118 further comprises a sheath 200 disposed around the proximal end 124 of the cabled electrical conductors 112 and provides the strain relief to the cable 109. An electrical isolator 203 is disposed within the sheath 200 and electrically connected to each of the cabled electrical conductors 112. The second electrical connector 127 is likewise disposed within the sheath 200 and electrically connected to each of the cabled electrical conductors 112 through the electrical isolator 203.

The electrical isolator 203 is an electrical isolation integrated circuit (“IC”) device and is mounted, in this particular embodiment, to a printed circuit board (“PCB”) 206. The PCB 206 is likewise disposed within the sheath 200 and is affixed to the cable electrical conductors 112. The PCB 206 includes a plurality of electrically conductive traces 207, shown in FIG. 2B, electrically connecting the cabled electrical conductors 112 to the electrical isolator 203 and the electrical isolator 203 to the second electrical connector 127. The electrical isolator 203 provides electrical isolation by capacitive coupling.

The illustrated embodiment employs an optional potting material 209 to provide thermal distribution of heat generated by the power consumption of the electrical isolator 203. However, the potting material 209 is but one means for distributing thermal energy disposed within the strain relief 118 and other embodiments may employ other means. Some embodiments may omit any means for distributing thermal energy.

Note that the strain relief 118 is generally longer and larger in diameter relative to conventional practice in order to accommodate the electrical isolator 203 and the PCB 206 of the MOPP. The actual strain relief provided by the strain relief 118 arises from the design and materials of the sheath 200. Note that the MOPP is removed from the medical host device 103 and is at the end of the cable 109 most distant from the sensor 106 and, hence, the patient (not shown).

FIG. 3 is a sectioned view of the cable first shown in FIG. 1 along line 3-3 in FIG. 1. In the illustrated embodiment, there are seven electrical conductors 112 although other embodiments may use other numbers of electrical conductors 112. Each electrical conductor 112 is insulated by a layer 300 of insulating material. The electrical conductors 112 are cabled by a cabling jacket 303, which may also be fabricated of an insulating material.

The claimed subject matter admits wide variation in the implementation of the host medical device 103. The host medical device 103 may be, for example, a patient monitor or patient monitoring device for monitoring and/or displaying physiological parameters of a patient, such as electrocardiogram (“EKG” or “ECG”), pulse rate, blood oxygen content, etc. Or, alternatively, the host medical device 103 may be, for another example, a patient therapy device such as a ventilator, an anesthesia machine, or a thermoregulation device (e.g., a warming therapy device). Those skilled in the art having the benefit of this disclosure may appreciate still other implementations for the host medical device 103 in various embodiments.

Implementations of the medical sensor 106 are similarly amenable to variation. For example, the medical sensor 106 may include only a single detector, such as a pulse oximeter, a thermometer, etc. Or, the medical sensor 106 may include multiple detectors, like an EKG lead set. Again, those skilled in the art having the benefit of this disclosure may appreciate still other implementations for the medical sensor 106 in various embodiments.

The first connector 115 and the second connector 127 of the illustrated embodiments are seven pin connectors. (Hence, the seven electrical conductors 112 in the cable 109.) However, the connectors may, in other embodiments, have more or fewer pins.

The electrical isolator 203 in the illustrated embodiments provides electrical isolation by capacitive coupling. Those in the art having the benefit of this disclosure will appreciate that the electrical isolation devices may alternatively employ optical or inductive coupling. Any of these techniques may be used for electrical isolation depending upon the embodiment.

Some embodiments of the patient care system 100 of FIG. 1 implement an Intelligent Patient Front End (“IPFE”) employing the cable disclosed herein. A new generation of physiological measurement devices, such as IPFE, can provide updated algorithms, features, and software updates for parameter measurement devices without corresponding releases of a new version of host monitor software. IPFEs, together with patient sensors, comprise a complete physiological patient parameter measurement delivery system. A number and a type of parameter measurement devices can be configured to meet varied and changing clinical needs. Remote access to versions, logs, self-tests, settings, history, and/or measurements via the Internet to one or more parameter measurement devices can provide a unified service approach.

One such IPFE system is disclosed in U.S. Pat. No. 11,721,939, commonly assigned herewith. The host medical device 103 may be an IPFE host device such as is described therein. The first connector 115 and the second connector 127 may be implemented using the connector designs disclosed in this patent in some embodiments. That connector is configured to electrically connect any two or more devices and provides an electrical connection that can be simply physically or tactually confirmed. However, embodiments of the claimed subject matter are not limited to such connectors and other connector designs may be used in other embodiments.

In some embodiments, the cable 100 of FIG. 1 may be implemented as what is known as a “smart cable”. Smart cables include processing elements that may be programmed to perform all manner of desired computational functions. These functions may range from data digitization to data conditioning, to device identification, and many others.

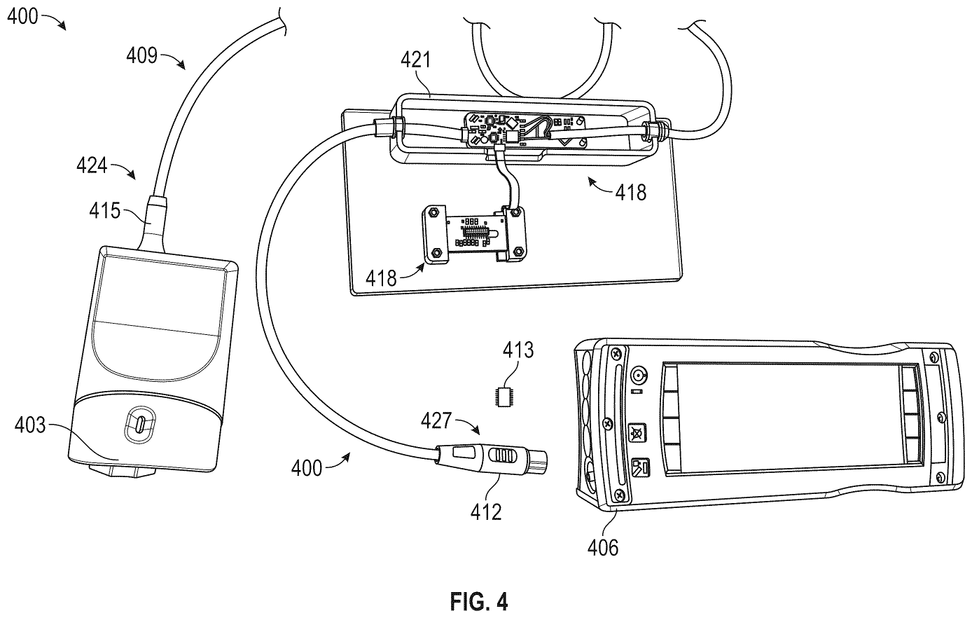

One such embodiment is shown in FIG. 4. FIG. 4 depicts one particular embodiment of a patient care system 400 in accordance with one or more embodiments. The patient care system 400 includes a medical sensor 403, a host medical device 406, and a cable 409. The cable 409 includes a strain relief 412 and a first connector 415 such as the strain relief 118 and the first connector 121, respectively, described above and shown in FIG. 1. Note that the second connector is disposed within the strain relief 412 and is therefore not directly visible in FIG. 4. The digital electrical isolator 413, however, is shown extracted from the strain relief 412. Also, the cable 409 is not shown connected to the host medical device 406 as it would be in operation.

The cable 409 is a smart cable and, so, includes processing elements 418. The processing elements 418 may be processors such as microprocessors, controllers, microcontrollers, erasable programmable read-only memories (“EPROMs”), electrically erasable programmable read-only memories (“EEPROMs”), application specific integrated circuits (“ASICs”). The processing elements 418 may also include as appropriate and/or desired memories, either on-ship or off-chip, read-only memories or random-access memories, volatile or non-volatile, etc., and combinations thereof. As shown, the processing elements 418 are disposed in a housing 421 and are spliced into the electrical conductors of the cable 409 intermediate the distal end 424 and the proximal end 427.

Accordingly, as described above, the present disclosure pertains to providing electrical isolation to a medical patient during medical care that is delivered, at least in part, by, with, or through medical devices. The disclosed technique provides a means of patient protection (“MOPP”) by disposing an electrical isolator in the strain relief of a cable providing electrical connectivity between a host medical device and one or more medical sensors. The strain relief and electrical isolator are disposed at an end of the cable that is most proximate to the host medical device. Electrical connectors are disposed on each end of the cable to provide connection with the host medical device and the one or more sensors.

Thus, in a first embodiment, a cable comprises a plurality of cabled electrical conductors, a first electrical connector, and a strain relief. The cabled electrical conductors have a first end and a second end. The first electrical connector disposed at the first end of the a plurality of cabled electrical conductors. The strain relief is disposed at the second end, includes a second electrical connector, provides electrical isolation to the first connector.

In a second embodiment, in the cable of the first embodiment, the strain relief comprises a sheath and an electrical isolator. The sheath is disposed around the second end of the cabled electrical conductors and provides the strain relief. The electrical isolator is disposed within the sheath and is electrically connected to each of the cabled electrical conductors. The second electrical connector is also disposed within the sheath and electrically connected to each of the cabled electrical conductors through the electrical isolator.

In a third embodiment, in the cable of the second embodiment, the electrical isolator is an electrical isolation integrated circuit device and the strain relief further comprises a printed circuit board on which the electrical isolation integrated circuit device and the second electrical connector are mounted and the cabled electrical conductors are affixed. The printed circuit board includes a plurality of electrically conductive traces electrically connecting the cabled electrical conductors to the electrical isolation integrated circuit device and the electrical isolation integrated circuit device to the second electrical connector.

In a fourth embodiment, in the cable of the first embodiment, the strain relief comprises a potting material providing thermal distribution.

In a fifth embodiment, in the cable of the first embodiment, the strain relief comprises means for distributing thermal energy.

In a sixth embodiment, in the cable of the first embodiment, at least one of the first electrical connector and the second electrical connector is a seven-pin connector.

In a seventh embodiment, in the cable of the first embodiment, the electrical isolation is provided by capacitive coupling.

In an eighth embodiment, the cable of the first embodiment is a smart cable including processing elements.

In a ninth embodiment, a patient care system comprises a host medical device, a medical sensor, and a cable electrically connecting the host medical device to the medical sensor. The cable comprises a plurality of cabled electrical conductors, a first electrical connector, and a strain relief. The cabled electrical conductors have a distal end and a proximal end relative to the host medical device. The first electrical connector is disposed at the distal end thereof and the strain relief disposed at the proximal end. The strain relief includes a second electrical connector and provides electrical isolation to the first connector.

In a tenth embodiment, in the patient care system of the ninth embodiment, the strain relief comprises a sheath and an electrical isolator. The sheath is disposed around the proximal end of the cabled electrical conductors and provides the strain relief. The electrical isolator is disposed within the sheath and is electrically connected to each of the cabled electrical conductors. The second electrical connector is also disposed within the sheath and electrically connected to each of the cabled electrical conductors through the electrical isolator.

In a eleventh embodiment, in the patient care system of the tenth embodiment, the electrical isolator is an electrical isolation integrated circuit device. The strain relief further comprises a printed circuit board on which the electrical isolation integrated circuit device and the second electrical connector are mounted and to which the cabled electrical conductors are affixed. The printed circuit board includes a plurality of electrically conductive traces electrically connecting the cabled electrical conductors to the electrical isolation integrated circuit device and the electrical isolation integrated circuit device to the second electrical connector.

In an twelfth embodiment, in the patient care system of the ninth embodiment, the strain relief comprises a potting material providing thermal distribution.

In a thirteenth embodiment, in the patient care system of the ninth embodiment, the strain relief comprises means for distributing thermal energy.

In a fourteenth embodiment, in the patient care system of the ninth embodiment, at least one of the first electrical connector and the second electrical connector is a seven-pin connector.

In a fifteenth embodiment, in the patient care system of the ninth embodiment, the electrical isolation is provided by capacitive coupling.

In a sixteenth embodiment, in the patient care system of the ninth embodiment, the host medical device is a patient monitoring device or a patient therapy device.

In a seventeenth embodiment, in the patient care system of the sixteenth embodiment, the patient monitoring device is an Intelligent Patient Front End device.

In an eighteenth embodiment, in the patient care system of the ninth embodiment, the cable is a smart cable including processing elements.

In a nineteenth embodiment, a method for providing electrical isolation to a patient in a patient care system comprises: connecting a cable at a first end thereof to a medical sensor; connecting the cable to a host medical device at a second end thereof opposite the first end; providing strain relief for the connection to the host medical device at the second end of the cable; and providing electrical isolation for the connection to the host medical device external to the host medical device and at the second end of the cable.

In a twentieth embodiment, in the method in the nineteenth embodiment, providing the electrical isolation includes capacitive coupling the connection at the second end to the connection at the first end.

In a twenty-first embodiment, in the method in the nineteenth embodiment, the cable comprises: a plurality of cabled electrical conductors; a first electrical connector electrically connected to the cabled electrical conductors at the first end of the cable; and a strain relief disposed at the second end of the cable, the strain relief including a second electrical connector and providing electrical isolation to the first connector.

In a twenty-second embodiment, in the patient care system of the twenty-first embodiment, the strain relief comprises a sheath and an electrical isolator. The sheath is disposed around the second end of the cabled electrical conductors and providing the strain relief. The electrical isolator disposed within the sheath and electrically connected to each of the cabled electrical conductors. The second electrical connector being disposed within the sheath and electrically connected to each of the cabled electrical conductors through the electrical isolator.

In a twenty-third embodiment, the method of the nineteenth embodiment further comprises processing acquired data in the cable.

In a twenty-fourth embodiment, a cable is substantially as shown and described herein.

In a twenty-fifth embodiment, a patient care system is substantially as shown and described herein.

In a twenty-sixth embodiment, a method for providing electrical isolation to a patient in a patient care system is substantially as shown and described herein.

The presently disclosed electrical isolation technique reduces the heat, power consumption, and cost of electrical patient isolation when providing isolation in the host medical device. Pushing the electrical isolation outside the host allows for increased efficiency. The technique does not incur isolation component cost, space, or power losses for unused ports in host parameter device. The space savings can be used to implement other, additional capabilities for the host medical device or reduce size and materials costs.

The disclosed technique also provides better heat management relative to conventional practice. The technique more evenly distributes power loss across the signal path, thereby spreading out the heat generated. The technique keeps heat away from the sensors and further away from the patient. Heat is kept outside of bulk air of the host medical device so that the electronics are protected from yet another heat source. Those in the art having the benefit of this disclosure may appreciate still further benefits and advantages of the technique claimed below.

The terms and words used in the description and claims herein are not limited to the bibliographical meanings but are merely used to enable a clear and consistent understanding of the present disclosure. Accordingly, it should be apparent to those skilled in the art that the following description of the present disclosure is provided for illustration purposes only, and not for the purpose of limiting the present disclosure as defined by the appended claims and their equivalents.

Features from different embodiments may be combined to form further embodiments, unless specifically noted otherwise. Variations or modifications described with respect to one of the embodiments may also be applicable to other embodiments. In some instances, well-known structures and devices are shown in block diagram form rather than in detail in order to avoid obscuring the embodiments.

Further, equivalent or like elements or elements with equivalent or like functionality are denoted in the following description with equivalent or like reference numerals. As the same or functionally equivalent elements are given the same reference numbers in the figures, a repeated description for elements provided with the same reference numbers may be omitted. Hence, descriptions provided for elements having the same or like reference numbers are mutually exchangeable.

It is to be understood that the singular forms “a”, “an”, and “the”, include plural referents unless the context clearly dictates otherwise. Thus, for example, reference to “a processor” or “a memory” includes reference to one or more of such processors or memories.

The expressions such as “include” and “may include” which may be used in the present disclosure denote the presence of the disclosed functions, operations, and constituent elements, and do not limit the presence of one or more additional functions, operations, and constituent elements. In the present disclosure, terms such as “include” and/or “have”, may be construed to denote a certain characteristic, number, operation, constituent element, component or a combination thereof, but should not be construed to exclude the existence of or a possibility of the addition of one or more other characteristics, numbers, operations, constituent elements, components or combinations thereof.

In the present disclosure, the expression “and/or” includes any and all combinations of the associated listed words. For example, the expression “A and/or B” may include A, may include B, or may include both A and B.

In the present disclosure, expressions including ordinal numbers, such as “first”, “second”, and/or the like, may modify various elements. However, such elements are not limited by the above expressions. For example, the above expressions do not limit the sequence and/or importance of the elements. The above expressions are used merely for the purpose of distinguishing an element from the other elements. For example, a first box and a second box indicate different boxes, although both are boxes. For further example, a first element could be termed a second element, and similarly, a second element could also be termed a first element without departing from the scope of the present disclosure.

The foregoing description, for purposes of explanation, used specific nomenclature to provide a thorough understanding of the disclosure. However, it will be apparent to one skilled in the art that the specific details are not required in order to practice the systems and methods described herein. The foregoing descriptions of specific examples are presented for purposes of illustration and description. They are not intended to be exhaustive of or to limit this disclosure to the precise forms described. Obviously, many modifications and variations are possible in view of the above teachings. The examples are shown and described in order to best explain the principles of this disclosure and practical applications, to thereby enable others skilled in the art to best utilize this disclosure and various examples with various modifications as are suited to the particular use contemplated. It is intended that the scope of this disclosure be defined by the claims and their equivalents below.

What is claimed is:

1. A cable, comprising:
a plurality of cabled electrical conductors, the cabled electrical conductors having a first end and a second end;
a first electrical connector disposed at the first end thereof; and
a strain relief disposed at the second end, the strain relief including a second electrical connector and providing electrical isolation to the first connector.

2. The cable of claim 1, wherein the strain relief comprises:
a sheath disposed around the second end of the cabled electrical conductors and providing the strain relief;
an electrical isolator disposed within the sheath and electrically connected to each of the cabled electrical conductors; and
a second electrical connector disposed within the sheath and electrically connected to each of the cabled electrical conductors through the electrical isolator.

3. The cable of claim 2, wherein
the electrical isolator is an electrical isolation integrated circuit device; and
the strain relief further comprises a printed circuit board on which the electrical isolation integrated circuit device and the second electrical connector are mounted and to which the cabled electrical conductors are affixed, the printed circuit board including a plurality of electrically conductive traces electrically connecting the cabled electrical conductors to the electrical isolation integrated circuit device and the electrical isolation integrated circuit device to the second electrical connector.

4. The cable of claim 1, wherein the strain relief comprises a potting material providing thermal distribution.

5. The cable of claim 1, wherein the strain relief comprises means for distributing thermal energy.

6. The cable of claim 1, wherein at least one of the first electrical connector and the second electrical connector is a seven-pin connector.

7. The cable of claim 1, wherein the electrical isolation is provided by capacitive coupling.

8. The cable of claim 1, wherein the cable is a smart cable including processing elements.

9. A patient care system, comprising:
a host medical device;
a medical sensor; and
a cable electrically connecting the host medical device to the medical sensor, the cable comprising:
a plurality of cabled electrical conductors, the cabled electrical conductors having a distal end and a proximal end relative to the host medical device;
a first electrical connector disposed at the distal end thereof; and
a strain relief disposed at the proximal end, the strain relief including a second electrical connector and providing electrical isolation to the first connector.

10. The patient care system of claim 9, wherein the strain relief comprises:
a sheath disposed around the proximal end of the cabled electrical conductors and providing the strain relief; and
an electrical isolator disposed within the sheath and electrically connected to each of the cabled electrical conductors,
the second electrical connector disposed within the sheath and electrically connected to each of the cabled electrical conductors through the electrical isolator.

11. The patient care system of claim 10, wherein
the electrical isolator is an electrical isolation integrated circuit device; and
the strain relief further comprises a printed circuit board on which the electrical isolation integrated circuit device and the second electrical connector are mounted and to which the cabled electrical conductors are affixed, the printed circuit board including a plurality of electrically conductive traces electrically connecting the cabled electrical conductors to the electrical isolation integrated circuit device and the electrical isolation integrated circuit device to the second electrical connector.

12. The patient care system of claim 9, wherein the strain relief comprises a potting material providing thermal distribution.

13. The patient care system of claim 9, wherein the strain relief comprises means for distributing thermal energy.

14. The patient care system of claim 9, wherein at least one of the first electrical connector and the second electrical connector is a seven-pin connector.

15. The patient care system of claim 9, wherein the electrical isolation is provided by capacitive coupling.

16. The patient care system of claim 9, wherein the host medical device is a patient monitoring device or a patient therapy device.

17. The patient care system of claim 16, wherein the patient monitoring device is an Intelligent Patient Front End (“IPFE”) device.

18. The patient care system of claim 9, wherein the cable is a smart cable including processing elements.

19. A method for providing electrical isolation to a patient in a patient care system, the method comprising:
connecting a cable at a first end thereof to a medical sensor;
connecting the cable to a host medical device at a second end thereof opposite the first end;
providing strain relief for the connection to the host medical device at the second end of the cable; and
providing electrical isolation for the connection to the host medical device external to the host medical device and at the second end of the cable.

20. The method of claim 19, wherein providing the electrical isolation includes capacitively coupling the connection at the second end to the connection at the first end.

21-26. (canceled)
